SEQUENCE LISTING (12 sequences)

SEQ ID NO: 1 - DNA, 18 bp, Artificial Sequence
  malB gene primer
gccatctcctgatgacgc

SEQ ID NO: 2 - DNA, 18 bp, Artificial Sequence
  MalB gene primer
atttaccgcagccagacg

SEQ ID NO: 3 - DNA, 40 bp, Artificial Sequence
  MalB gene primer
ctggggcgaggtcgtggtattccgacaaacaccacgaatt

SEQ ID NO: 4 - DNA, 42 bp, Artificial Sequence
  MalB gene primer
cattttgcagctgtacgctcgcagcccatcatgaatgttgct

SEQ ID NO: 5 - DNA, 24 bp, Artificial Sequence
  MalB gene primer
ctttgtaacaacctgtcatcgaca

SEQ ID NO: 6 - DNA, 25 bp, Artificial Sequence
  MalB gene primer
atcaatctcgatatccatgaaggtg

SEQ ID NO: 7 - DNA, 22 bp, Artificial sequence
  STY1607 primer
gacttgcctttaaaagatacca

SEQ ID NO: 8 - DNA, 19 bp, Artificial Sequence
  STY1607 primer
agagtgcgtttgaacactt

SEQ ID NO: 9 - DNA, 38 bp, Artificial Sequence
  STY1607 primer
cctggggccaaatggcattatgcactaagtaaggctgg

SEQ ID NO: 10 - DNA, 40 bp, Artificial Sequence
  STY1607 primer
aacttgctgctgaagagttggaccgaatgactcgaccatc

SEQ ID NO: 11 - DNA, 18 bp, Artificial Sequence
  STY1607 primer
tcggatggcttcgttcct

SEQ ID NO: 12 - DNA, 25 bp, Artificial Sequence
  STY1607 primer
caagggtttcaagactaagtggttc

(19) United States
(12) Patent Application Publication
(10) Pub. No.: US 2021/0180116 A1
(43) Pub. Date: Jun. 17, 2021

(54) ASSAY USING MULTI-LAYER MEMBRANE TO DETECT MICROBIOLOGICAL TARGET AND METHOD OF MANUFACTURING MULTI-LAYER MEMBRANE

(71) Applicant: CALIFORNIA INSTITUTE OF TECHNOLOGY, Pasadena, CA (US)

(72) Inventors: Michael R. Hoffmann, South Pasadena, CA (US); Xingyu Lin, Pasadena, CA (US); Xiao Huang, Pasadena, CA (US)

(21) Appl. No.: 17/270,395

(22) PCT Filed: Aug. 28, 2019
(86) PCT No.: PCT/US2019/048546

§ 371 Date: Feb. 22, 2021

Related U.S. Application Data
(60) Provisional application No. 62/724,469, filed on Aug. 29, 2018.

(51) Int. Cl. C12Q 1/6806; C12M 1/26; C12Q 1/6844; C12Q 1/689

(52) U.S. Cl. CPC C12Q 1/6806; C12Q 1/689; C12Q 1/6844; C12M 33/14

(57) ABSTRACT

A membrane, method and system are disclosed for rapid, sensitive and precise detection of an agent suspected of being present in a sample. The agent may be a cell or microorganism, e.g., a single pathogenic bacteria, and the sample may be small, e.g., milliliters of unprocessed environmental water. The sample is processed by filtering it through an asymmetric membrane having multiple layers. One layer has microchannels for capturing the agent and another layer has nanochannels for passing particles smaller than the agent. Amplification reagents, such as loop-mediated isothermal amplification (LAMP) reagents, are load onto membrane so that the microchannels act as nanoreactors, creating quantifiable amplicons within the pores on the exposed surface of the membrane in response to captured agent. The amplicons may be imaged and counted using a fluorescent camera. The membrane is capable of agent capture, concentration, purification, partition, lysis and digital LAMP without off-membrane sample treatments.

### CROSS REFERENCE TO RELATED APPLICATIONS

This Application claims the benefit of U.S. Provisional Patent Application Ser. No. 62/724,469, filed on Aug. 29, 2018, which is incorporated by reference herein in its entirety.

### TECHNICAL FIELD

The present disclosure generally relates to techniques for detecting a microbiological agent of interest in a sample, for example, techniques for detecting or monitoring target microorganisms in environmental water samples.

### BACKGROUND

Intestinal parasitic infections and diarrheal diseases, which are caused by waterborne pathogens, have become a leading cause of morbidity and mortality, owing to insufficient hygiene and poor sanitation. Globally, more than 2.2 million people die each year because of waterborne pathogen infections with a resulting economic loss of nearly 12 billion US dollars annually worldwide. Given the low infectious dose of many waterborne pathogens, the presence of even a single bacterium in the environment may pose a serious health risk. According to the US Environmental Protection Agency (EPA), the concentration of Escherichia coli (E. coli) and Enterococci in environmental recreational samples should be less than 1.26 and 0.35 CFU/mL (colony-forming units/mL), respectively. These strict standards require a detection method that is not only ultrasensitive, but also quantitative and precise.

Historically, culture-based detection methods have been the gold standard for bacteria identification and titration, although they typically require days to obtain the results and usually do not differentiate bacteria at the species levels. Quantitative real-time polymerase chain reactions (PCRs) can shorten that time to several hours, but it requires expensive instrumentation and is poorly suited for absolute quantification in certain circumstances.

Droplet-based microfluidics have emerged as promising methods for digital cell quantification, as well as single cell heterogeneity analysis. Generally, with these techniques, each cell is encapsulated into an individual droplet, and the specific cell information (e.g., specific DNA, RNA, proteins, enzymes, metabolism, and/or antibodies) are converted into a fluorescence signal, and thus, enable direct counting. This “digital format” allows simple, rapid, and multiplexed detection of specific cell strains in the samples from commensal ones. However, the concentration of pathogenic bacteria in environmental samples is may be so low in some cases that it is beyond the detection limit of most microfluidic devices due to their limitation of using microliter samples.

To detect bacteria having low concentrations, for example, concentrations of less than one cell per mL, at least several milliliters of samples should be analyzed, no matter how sensitive the detection method is. For most microfluidic chips, it may take several hours or days for bulk sample loading (even more for nanofluidic chips), which may be impractical in terms of time and precious bioreagents and may also inactivate biochemical reactions. In addition, multiple pre-treatment steps of the sample are still typically required for raw samples, in order to remove inhibitors, exclude particles, enrich bacteria, or extract DNA before ultimate analysis. Furthermore, the access to microfluidics, especially nanofluidics, typically calls for elaborate chip fabrication and sophisticated fluid control (e.g., pump, vacuum, centrifuge, valve and the like), limiting their accessibility to users without related expertise and instruments.

Accordingly, a rapid, simplified, low-cost bioassay for detecting/quantifying biological targets, e.g., microbes, is desirable, particularly one that is suitable for point-of-use field testing of environmental waters in places with limited resources, or for point-of-care users or scientific or medical laboratories to perform bioassays in an inexpensive and simplified way.

### SUMMARY

Disclosed herein are assay methods and systems that employ a multi-layer, asymmetric membrane that allows field testers, point-of-care users or laboratories to perform digital quantification, single cell analysis, or other bioassays in an inexpensive, fast, flexible, and simplified way. The simple and low-cost analysis platform described herein has an enormous potential for the detection of pathogens, exosomes, stem cells, and viruses as well as single cell heterogeneity analysis in environmental, food, and clinical research.

In accordance with exemplary embodiments, one or more methods and systems are provided for detecting a target microbiological agent, e.g., a cell, microorganism or target nucleic acid, in a sample suspected of containing the target agent. The sample may generally include one or more fluids, gases, or solids, or any combination of the foregoing, capable of being successfully filtered by the asymmetric membrane system.

In accordance with an exemplary embodiment, a method is provided for detecting a target agent in a sample. The method includes filtering the sample to remove particles larger than the target agent to produce a filtered sample. The filtered sample is then passed through a membrane having first channels forming corresponding pores on an exposed surface of the membrane. The pores admit the sample into the first channels. Each of the first channels has a predetermined width configured to admit a predetermined number of the target agent into each of the first channels. Sample output from the first channels pass through second channels formed in the membrane and connected to the first channels. The second channels are configured to trap one or more individuals of the target agent in the first channels and allow other constituents of the filtered sample to pass through the second channels and out of the membrane. After the sample is filtered through the membrane, one or more reagents are placed into the first channels through the pores to cause an amplification reaction involving the target agent individuals trapped in the first channels. The target agent is detected by the presence or absence of one or more amplification products that may form in the first channels, resulting from the amplification reaction amplifying a nucleic acid of the target agent, if the target agent is present in any of the first channels. The presence of the amplification products is indicative of the presence of the target agent in the sample and the absence of the amplification products is indicative of the absence of the target agent in the sample. Concentration of the target agent in the sample may be determined based on the number of fluorescent amplification products (e.g., amplicon dots) appearing in the pores on the exposed surface of the membrane after the reaction. The amplicons may be imaged using a smartphone or a fluorescent microscope.

In accordance with another exemplary embodiment, the membrane for capturing the target agent includes a first layer having first channels passing therethrough. The first channels form corresponding pores on an exposed surface of the first layer for admitting the sample into the first channels. Each of the first channels has a predefined width configured to admit a number of the target agent into each first channel. The membrane includes a second layer that has an asymmetrical number and size of channels when compared to the first layer. The second layer contacts first layer so that second channels passing therethrough connect with the first channels of the first layer. The second channels are configured to retain one or more individuals of the target agent in the first channels and pass one or more other constituents of the sample out of the membrane though an exposed surface of the second layer.

In accordance with a further exemplary embodiment, a method of manufacturing a composite, asymmetrical membrane for detecting a target agent includes providing a first track-etched membrane having first channels passing therethrough between a first surface of the first membrane and a second surface of the first membrane. Each of the first channels has a predetermined width configured to admit a predetermined number of the target agent into each of the first channels. A second track-etched membrane is placed on either the first surface or second surface of the first track-etched membrane. The second track-etched membrane has second channels passing therethrough. Each of the second channels has a width smaller than the predetermined width of the first channels. With the second track-etched membrane emplaced on the first track-etched membrane, the first and second track-etched membranes are heated to bond them together, whereby forming the composite membrane.

The foregoing summary does not define the limits of the appended claims. Other aspects, embodiments, features, and advantages will be or will become apparent to one with skill in the art upon examination of the following figures and detailed description. It is intended that all such additional features, embodiments, aspects, and advantages be included within this description and be protected by the accompanying claims.

### BRIEF DESCRIPTION OF THE FIGURES

It is to be understood that the drawings are solely for purpose of illustration and do not define the limits of the appended claims. Furthermore, the components in the figures are not necessarily to scale. In the figures, like reference numerals designate corresponding parts throughout the different views.

FIG. 1 is a cross-sectional schematic illustration of an exemplary asymmetric membrane for filtering a sample.

FIG. 2 is cross-sectional schematic illustration of an exemplary asymmetric membrane system, including a pre-filter, shown filtering a sample.

FIG. 3 is perspective schematic illustration of the exemplary asymmetric membrane system of FIG. 2, shown filtering a sample.

FIG. 4 is cross-sectional schematic illustration of an exemplary asymmetric membrane system placed in a filter holder for filtering a sample.

FIG. 5 is a process diagram illustrating an exemplary method of fabricating an asymmetric membrane.

FIG. 6 is a flowchart diagram illustrating an exemplary method of detecting an agent with the asymmetric membrane.

FIG. 7 is a schematic illustration showing components of an exemplary asymmetric membrane assay system or kit.

FIGS. 8a-e are images of an exemplary asymmetric membrane prepared according to the method of FIG. 5.

FIG. 9a-d are scanning electron microscope (SEM) images of the top surfaces of exemplary asymmetric membranes having various micropore and nanopore widths.

FIG. 10 is a perspective-view SEM image of an exemplary asymmetric membrane prepared according to the method of FIG. 5.

FIG. 11a is a top-down view SEM image of an exemplary loaded asymmetric membrane that has completed filtration.

FIGS. 11b-c are graphs of example simulation and experimental results showing the distribution of a target agent in micropores of an example membrane (FIG. 10b) and permeation of the target agent into the micropores of a membrane as a function of the width of the micropores (FIG. 10c).

FIGS. 12a-f are graphs of example experimental results showing certain performance characteristics of an exemplary asymmetrical membrane system.

### DETAILED DESCRIPTION

The following detailed description, which references to and incorporates the drawings, describes and illustrates one or more examples of assay systems, kits and methods to detect and/or quantify the presence of a target agent in a sample. These examples, offered not to limit but only to exemplify and teach embodiments of inventive assays, membranes, methods, kits and systems, are shown and described in sufficient detail to enable those skilled in the art to practice what is claimed. Thus, where appropriate to avoid obscuring the invention, the description may omit certain information known to those of skill in the art. The disclosures herein are examples that should not be read to unduly limit the scope of any patent claims that may eventual be granted based on this application.

The word “exemplary” is used throughout this application to mean “serving as an example, instance, or illustration.” Any system, method, device, technique, feature or the like described herein as “exemplary” is not necessarily to be construed as preferred or advantageous over other features.

As used in this specification and the appended claims, the singular forms “a,” “an,” and “the” may include plural referents unless the content clearly dictates otherwise.

Although any methods and materials similar or equivalent to those described herein can be used in the practice or testing of the invention(s), specific examples of appropriate materials and methods are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

As used herein, a “micropore” or “microchannel” (alternately referred to herein as “micropore” or a “microchannel”) refers to an opening, orifice, gap, conduit, passage, chamber, or groove in a membrane/layer, where the micropore or microchannel is of sufficient dimension that allows passage or analysis of at least a single target agent (e.g., a cell, bacteria, virus, biological particle, microbe, or the like). In some embodiments, a micropore can allow passage or admit more than one target agent. As used herein, “micro” generally refers to micrometer scale dimensions.

As used herein, a “nanopore” or “nanochannel” (alternately referred to herein as “nanopore” or a “nanochannel”) refers to an opening, orifice, gap, conduit, passage, chamber, or groove in a membrane/layer, where the nanopore or nanochannel is of dimension or configuration that prevents passage of a single target agent. As used herein, “nano” generally refers to nanometer scale dimensions.

As used herein, “pore size” generally refers to the width of a micropore or nanopore, unless the context indicates otherwise. In some embodiments, “micro” refers to micrometer scale dimensions.

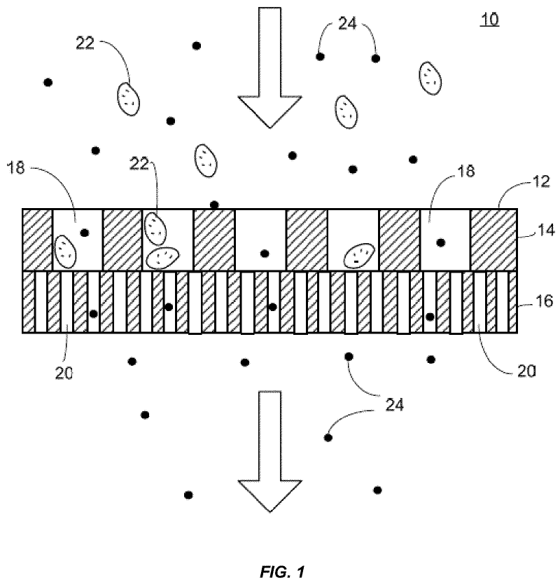

FIG. 1 is a cross-sectional schematic illustration of an exemplary asymmetric, substantially planar membrane 12 in a filtering environment 10, shown filtering a sample that includes a target agent 22 and particles 24 that are smaller than the target agent 22. As shown by the arrows of FIG. 1, the sample flows from the top of the diagram, through the membrane 12, and toward the bottom of the Figure. In the example shown, the membrane 12 includes two layers 14, 16 in contact with each other. Other embodiments of the membrane may include more than two layers. The first or top layer 14 includes micropores 18 that have a width sufficient to admit individuals of the target agent 22 into the microchannels 18. The microchannels 18 pass through the top layer 14 from the top, exposed surface of the membrane 12 to the bottom, exit surface of the layer 14. The bottom or second layer 16 includes nanochannels 20 that may be located proximate to the microchannels 18 such that the microchannels 18 and many of the nanochannel 20 are in fluidic contact. The nanochannels 20 are sized and/or configured to prevent passage of the target agent 22 through nanochannels 20 and out of the membrane 12, and instead capture the agent 22 in the microchannels 18. The nanochannels 20 pass through the bottom layer 16 from the top surface of the bottom layer 16 to the bottom, exit surface of the membrane 12.

As shown, the microchannels 18 and nanochannels 20 may be generally aligned with each other, for example in vertical alignment. However, in other embodiments, such alignment is not necessary. For example, the microchannels 18 and nanochannels 20 may be at an angle relative to each other or curved.

Although certain exemplary embodiments disclosed herein may focus on the target agent being microbial pathogens (e.g., E. coli, Salmonella) in environmental water, the disclosure is not limited to these agents or samples. In other embodiments, the target microbiological agent may be any suitable cell, microorganism or target nucleic acid, in a sample suspected of containing the target agent. The sample may generally include one or more fluids, gases, solids, or mixtures, or any combination of the foregoing, capable of being successfully filtered by the asymmetric membrane 12. In some embodiments, the sample may be prepared prior to filtration by the asymmetric membrane (e.g., culturing a sample to allow growth of microbes before filtration and the like).

The asymmetric membrane 12 is a novel and robust nanofluidic platform that may be used, for example, for digital detection of single pathogenic bacteria directly in a relatively small sample, e.g., 10 mL or less of unprocessed environmental water samples. The asymmetric membrane 12 is asymmetric in the sense that it may have uniformly sized micropores 18 on one side (top layer 14), and a high density of vertically aligned nanochannels 20 on the other side (bottom layer 16).

When used to process a sample to detect a target agent, the membrane 12 may cover the processing steps from sample concentration, purification, and partition to a final amplification reaction to detect the agent, e.g., digital loop-mediated isothermal amplification (LAMP), as disclosed herein. By filtration with the membrane 12, bacteria or other targets may be enriched and partitioned inside the micropores 18, while inhibitors or particles smaller than the target agent, which are typically found in samples such as environmental waters, (e.g., proteins, heavy metals and organics) may be washed away through the nanochannels 20.

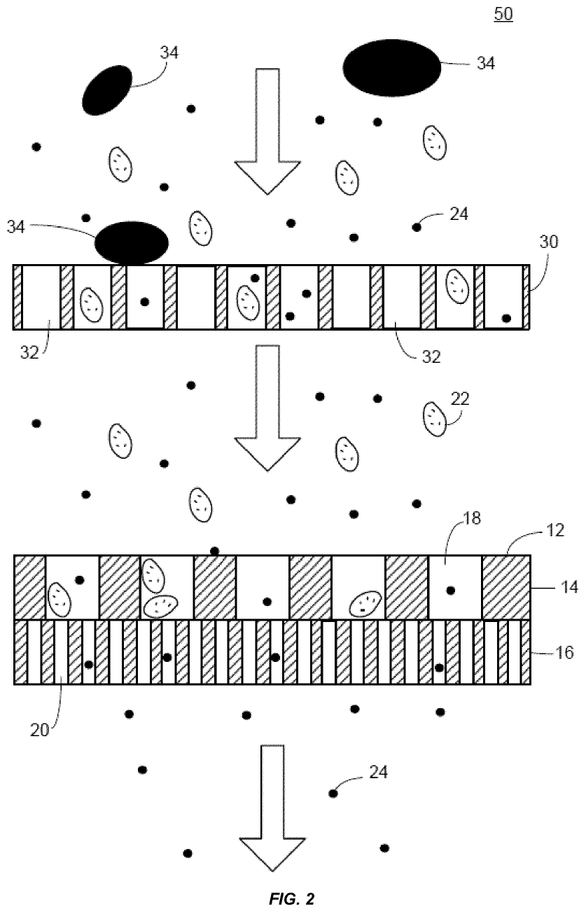

To remove particles larger than the target agent 22 in the sample, a sacrificial filter 30 (e.g., a pre-filter) may be placed before the membrane 12 in the sample flow. FIG. 2 is cross-sectional schematic illustration of an exemplary asymmetric membrane system 50, including the pre-filter 30. As shown by the arrows of FIG. 2, the sample flows from the top of the diagram, through the filter 30 and then the membrane 12, and toward the bottom of the Figure. The filter 30 includes microchannels 32 that are sized so that their width is sufficient to pass the target agent 22, but block larger particles 34. The larger particles 34, e.g., indigenous plankton, positively charged pollutants, algae, solid particles, or the like, in the sample may be excluded by using the sacrificial filter 30, which may be a microchannel membrane stacked on top of the asymmetric membrane 12.

The system 50 may be suitable for processing complex environmental samples, where the presence of various large particles and organisms could easily block the asymmetric membrane 12 or inhibit the following enzyme-driven nucleic-acid amplification processes. In some embodiments, the pre-filter 30 may be a sacrificial track-etched polycarbonate (PC) membrane with uniform microchannels and negatively charged microchannel surfaces stacked above the asymmetric membrane 12 for sample pre-filtration. The function of this sacrificial layer 30 is to exclude all large particles and adsorb positively charged matters, but not obstructing the passage of target agent 22.

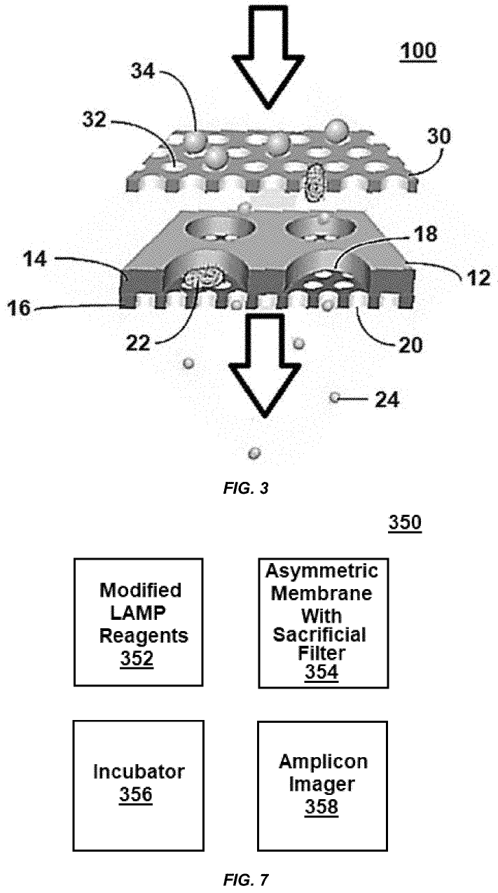

FIG. 3 is perspective-view schematic illustration 100 of the exemplary asymmetric membrane system 50 of FIG. 2. The system 50 is shown filtering a sample containing the target agent 22, smaller particles 24, and larger particles 34.

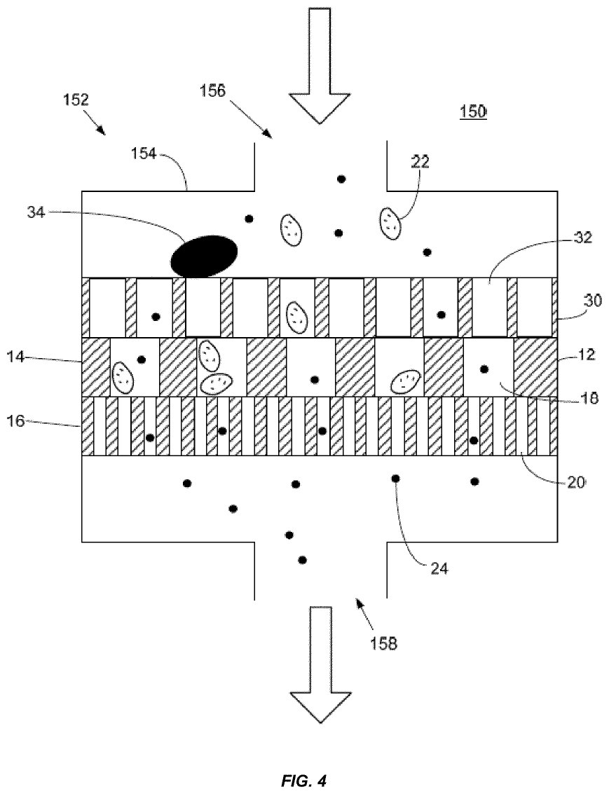

FIG. 4 is cross-sectional schematic illustration of an exemplary asymmetric membrane filter system 150, with the membrane system 50 placed in a filter holder 154 for filtering a sample. The filter holder 154 includes an inlet 156 for admitting the sample and an outlet 158 for passing out the sample filtrate. The filter holder 154 is a container that generally encapsulates and supports the membrane system 50 in place so that substantially all of the sample passes through the membrane system 50 during the filtering process to capture the target agent on the membrane 12. The filter holder 154 may be a commercially available filter holder, e.g. those available from Swinnex of Kent, Wash., that can be opened and closed so that the membrane system 50 is removably placed in the holder 154. The holder 154 may include two or more removably attached pieces so that the membrane system 50 can be inserted into or removed from the holder 154. The holder 154 may also include internal supports (not shown) for holding the membrane system 50 in place and preventing tears, such as a permeable wire or plastic mesh located below the membrane system 50.

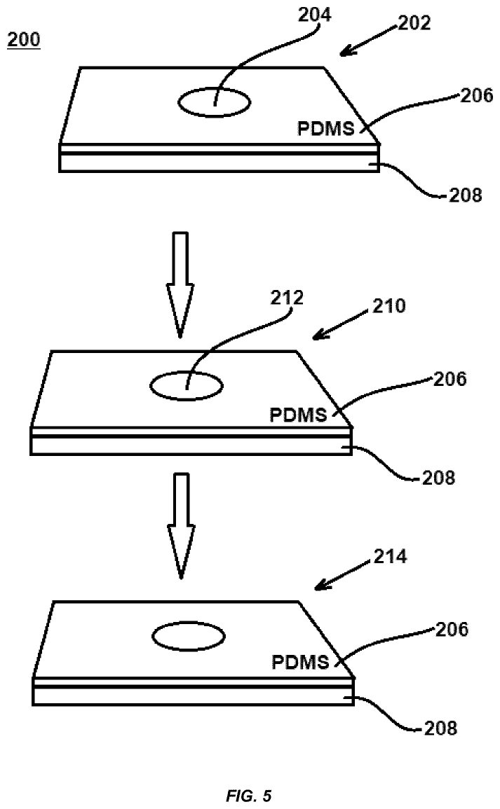

FIG. 5 is a process diagram illustrating an exemplary method 200 of fabricating an asymmetric membrane. Different techniques may be used to make the asymmetric membranes disclosed herein, such as asymmetric etching, asymmetric modification, or asymmetric combination. However, a novel, inexpensive and robust method 200 for the preparation of asymmetric membranes is illustrated by FIG. 5. The method 200 utilizes symmetric track-etched membranes, for example, commercially-available track-etched membranes made of a polymer or any other suitable material, for example, polyethylene terephthalate (PET), polyester, or polycarbonate (PC) films. The polymer membranes may have a thickness of between 5 to 25 microns, and in some embodiments, greater than 25 microns and in others less than 5 microns.

Track-etched membrane technology is an example of industrial application of track etching technique. Track-etched membranes offer distinct advantages over conventional membranes due to their precisely determined structure. Their pore size, shape and density can be varied in a controllable manner so that a membrane with the required transport and retention characteristics can be produced.

The main differences between track-etched membranes and traditional membranes are the correct geometry of pores, ability to control their number per unit of membrane surface area and narrow pore size (width) distribution. Pore shape can be cylindrical, conical, funnel-like, or cigar-like. The pore sizes of track-etched membranes may be in the range from 1 nm to 100 s of micrometers (track-etched nano and micro-filtration membranes, respectively).

Referring now to FIG. 5, in step 202 a track-etched membrane 204 having micropores is placed on top of a polydimethylsiloxane (PDMS) sheet 206 contacting a heating element 208, such as a hotplate. A PDMS film may be used to prevent thermal deformation of the membranes at high temperature. PDMS films may be prepared by mixing their precursor and curing agent at a ratio of 10:1 and heating the mixture to 75° C. for 1.5 hours. Other non-reactive sheets other than a PDMS sheet may be used, and other heating elements such as a radiant heat source, e.g., infrared, may be used in other embodiments.

In step 210, a second track-etched membrane 212 having nanopores is placed on top of the micropore membrane 204.

In step 214, the stacked track-etched membranes 204, 212 are heated to bond them together. This results in an asymmetric membrane having numerous vertically-aligned microchannels and nanochannels.

In some embodiments, commercial PC membranes may be coated with polyvinylpyrrolidone (PVP). This hydrophilic coating may be removed first, prior to step 202, since it may affect amplification reactions using the membrane, e.g., LAMP reactions. PVP removal may be accomplished by dipping membranes in 10% acetic acid for 60 minutes, followed by heating to 120° C. for 30 minutes, prior to performing the above method steps.

In an example embodiment of the method, two symmetric track-etched polycarbonate (PC) membranes, e.g., commercially-available track-etched PC membranes from Sterlitech Corporation of Kent, Wash., may be stacked together on the PDMS sheet 206 and then heated at 165° C. by a hotplate for about one minute, ±10 seconds. The first membrane 204 may have micropores of 25 μm and the second membrane 212 may have nanopores of 400 nm. The two membranes are then removed from the PDMS heating element. After the short heating duration, the two membranes are irreversibly bonded together.

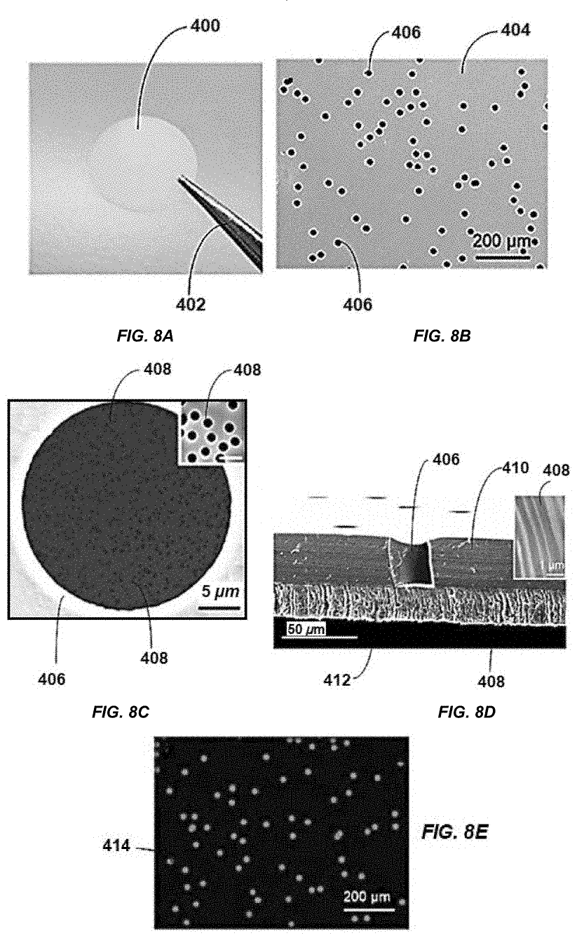

FIG. 8a shows a photograph of an example asymmetric PC membrane 400 made according to this process held by tweezers 402. The asymmetric membrane 400 exhibited excellent sealing between the two membrane layers. The bonding mechanism between the two membrane layers 204, 212 may be attributed to the glass transition properties of the thermoplastic material. Polycarbonate has a glass transition temperature of ˜150° C. Above this temperature, the micropore and nanopore membranes undergo a transition from a glassy state to a rubbery state, where they become soft while the micro/nanostructure remains unchanged. The long-range motion of the polymer chains in the rubbery state, facilitates the tight adhesion of two membranes. Thus, the two layers may be held tightly together by glass-transition-induced bonding.

FIG. 8b shows a top-view scanning electron microscopy (SEM) image of the asymmetric membrane 400, confirming the presence of uniform micropores 406 on its top surface 404. Top-view and cross-sectional view SEM images disclosed herein were obtained on with a ZEISS 1550VP field emission scanning electron microscope (FESEM). The thickness of each membrane layer was about 25 microns. The micropore size was measured to be 25 μm wide and the pore density was about 104 pores/cm². The pore width size was uniform (25 μm, ±10%). Magnification of the image of FIG. 8b reveals the high density of nanochannels 408, with uniform diameters of 400 nm ±10%, within each micropore 406 (FIG. 8c). FIG. 8c is a high-magnification top-view SEM image of one micropore 406 of the example asymmetric membrane 400. The inset of FIG. 8c shows the magnified image with a scale bar of 1 μm.

Compared to the original micropore and nanopore PC membranes 204, 212, the morphology of micropores and nanochannels of the asymmetric membrane remains generally unchanged after the heat treatment. FIG. 8d is a cross-sectional view SEM image of the example asymmetric membrane 400, showing the presence of micropores 406 on the top membrane layer 410, and vertically aligned nanochannels 408 in the bottom membrane layer 412. The two membranes 410, 412 are bonded tightly together without any gap, as a result of the fabrication method. A strong bonding is advantageous for the asymmetric membrane 400 to prevent it from splitting during filtration with applied pressure. The successful sealing and parallel perpendicular nanochannels 412 ensure the isolation of each pore 406 and prevent cross-contamination between pores.

The wettability of PC membranes 410, 412 before and after thermal treatments was tested. The contact angle of an example LAMP solution on PC membranes was increased slightly after thermal bonding of the two membrane layers, from 40±3° to 50±2° for membranes with 25 μm pore size, and from 47±3° to 54±4° for membranes with 400 nm pore size. The low contact angles indicate that solutions can easily enter the micropores and nanochannels of the PC asymmetric membrane.

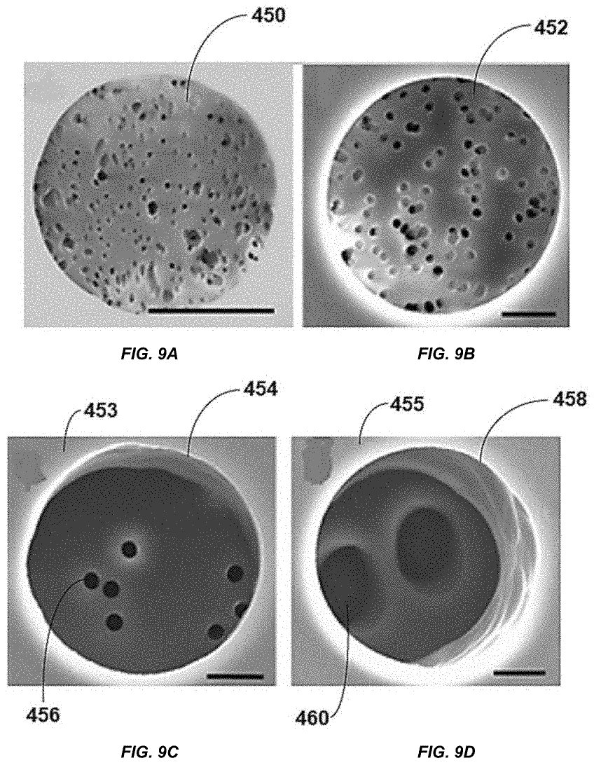

In addition to the above disclosed pore sizes and materials, other embodiments of the asymmetric membrane may be successfully prepared using other combinations of pore size (range from 200 nm to 30 μm) and other materials (polyester or PET). FIGS. 9a-d are top-down view SEM images of other asymmetric membranes 450, 452, 453, 455 that were prepare in accordance with the fabrication methods disclosed herein. The scale bars is each image are 5 μm. FIG. 9a is an SEM image of an example asymmetric PC membrane 450 prepared with a micropore width of 10 μm and nanopore width of 200 nm. FIG. 9b is an SEM image of an example asymmetric PC membrane 452 prepared with a micropore width of 25 μm and nanopore width of 1 μm. FIG. 9c is an SEM image of an example asymmetric PC membrane 453 prepared with a micropore 454 width of 25 μm and nanopore 456 width of 2 μm. FIG. 9d is an SEM image of an example asymmetric PC membrane 455 prepared with a micropore 458 width of 25 μm and nanopore 460 width of 8 μm.

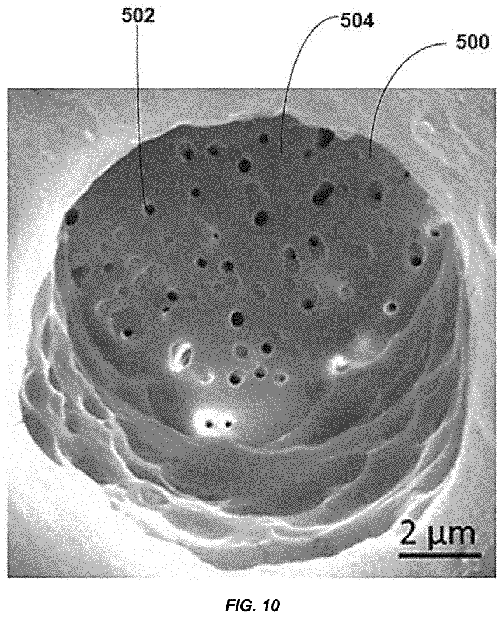

FIG. 10 is a perspective-view SEM image of another exemplary asymmetric membrane 500 prepared according to the method of FIG. 5. The membrane 500 has a top layer exposed surface 504 having a plurality of micropores 502 formed therein. In accordance with some embodiments, the asymmetric membrane 500 is composed of different materials layered together. The top membrane layer is a commercially-available track-etched polyester membrane with 10 μm width pore size, and the bottom membrane layer is a commercially available polycarbonate track-etched membrane with 400 nm width nanopore size.

The exemplary asymmetric membranes disclosed herein with relatively large micropores on one side and high-density nanochannel-arrays on the other side may function as nanofluidics for digital target agent counting, e.g., bacteria counting. To successfully achieve this functionality, some embodiments of the asymmetric membrane may have the following features: (i) the microchannels and nanochannels may each have a uniform width and may be vertically aligned with each other, without horizontal interconnections between the channels for isolation; (ii) the micropores on one side of asymmetric membrane may be wide enough (e.g., greater than 20 μm) for visual counting, while the nanochannels in other side may be less than 400 nm for bacteria capture within the microchannels; (iii) a strong bonding is necessary between the microchannel and nanochannel layer to prevent leakage around the microchannels; (iv) to enable rapid filtration, which may be done manually, a high density of nanochannels may be present to lower the applied pressure and increase the flow rate through the membrane; and (v) the asymmetric membrane should possess excellent mechanical/chemical/thermal stability.

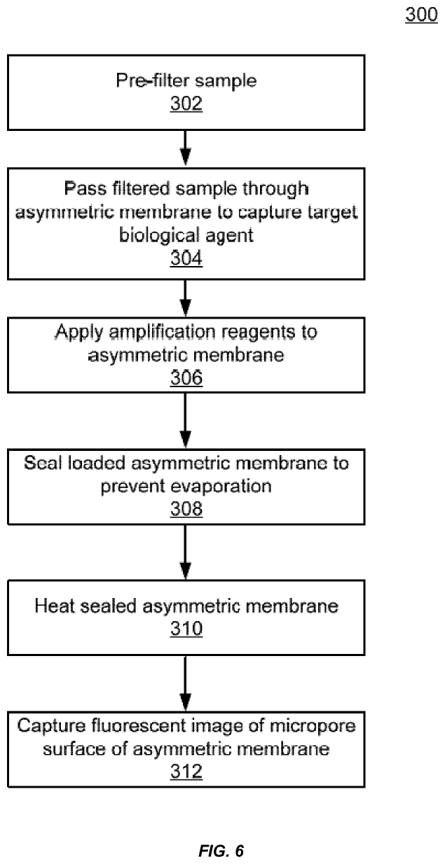

FIG. 6 is a flowchart diagram illustrating an exemplary method 300 of detecting/quantifying a target agent with the asymmetric membrane system.

In step 302, a sample suspected of containing a target agent is filtered to remove impurities and particles larger than the target agent, so that they do not clog the micropores. A sacrificial filter may be used to perform this step. Other filtering techniques may alternatively be employed. During the pre-filtration, large particles and positively charged pollutants may be removed by the sacrificial pre-filter placed in front of the asymmetric membrane, while target agent particles can pass through and then concentrate inside the micropores of the membrane.

In step 304, the pre-filtered sample is then passed through an asymmetric membrane, such as any of those disclosed herein. Individuals of the target agent may be captured in the microchannels of the membrane. Particles smaller than the target, such as small inhibitors typically found in environmental samples, such as proteins, humic acids, organics, and heavy metals or any combination thereof, are passed through the nanochannels of the membrane and washed away. The sample may be pushed through the asymmetric membrane using any suitable means, for example, using a syringe pushed by hand or an electric pump.

Next, in step 306, amplification reagents are applied into the microchannels of the membrane for an amplification reaction. The reagents may be applied as a mix in small quantities, e.g., about 25 μL, by using a conventional handheld applicator. Each microchannel of the membrane may function as an individual nanoreactor for single DNA amplification of the target agent. In some embodiments, the amplification reaction is selected from the group consisting of polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, thermal asymmetric interlaced PCR (TAIL-PCR).

In some embodiments, the amplification reaction is selected from the group consisting of Loop-Mediated Isothermal Amplification (LAMP), reverse transcription-LAMP (RT-LAMP), modified LAMP or modified RT-LAMP reaction, Helicase-Dependent Amplification (HDA), Rolling Circle Amplification (RCA), Multiple Displacement Amplification (MDA), Recombinase Polymerase Amplification (RPA), or Nucleic Acid Sequence-Based Amplification (NASBA). In accordance with some embodiments, a modified LAMP or modified RT-LAMP reaction may have reagents that include NaF and/or lysozyme.

In step 308, the loaded asymmetric membrane may then be optionally sealed to prevent evaporation. The wetted membrane may be sealed between two PDMS films to remove residual reagents from the membrane expose top surface. The PDMS films may be prepared as described elsewhere herein.

In step 310, the sealed membrane is optionally heated sufficiently to incubate an amplification reaction involving the target agent. During incubation, each pore of the asymmetric membrane functioned as an individual nanoreactor for template amplification, generating a bright fluorescence if a target agent is present inside a microchannel. In order to prevent water evaporation, the top piece of PDMS may be peeled off the membrane after incubation, followed by addition of mineral oil to cover the whole top surface of the membrane.

Finally, in step 312, the amplification products resulting from the amplification reaction in the micropores are detected and may be quantified to determine a concentration of the target agent in the sample. The amplification products (i.e., amplicons) may be spotted by taking a fluorescent image of the exposed micropore surface to the membrane using a camera or fluorescent microscope. The quantification of the target agent may be based on the volume of the sample filtered through the membrane and the number of target agent individuals detected in the micropores of the membrane following the amplification reaction. FIG. 8e shows an example fluorescent image 414 have visible amplification products in the micropores of an example membrane having undergone a sample filtration and amplification reaction, in accordance with the method of FIG. 6.

FIG. 7 is a schematic illustration showing components of an exemplary asymmetric membrane assay system or kit 350, which may enable microbial pathogen quantification, e.g., bacterial quantification, within about one hour using an asymmetric membrane system disclosed herein and standard laboratory devices. The system 350 may be a kit that includes amplification reagents, such as the modified LAMP reagents 352 disclosed herein; one or more asymmetric membranes, e.g., those disclosed herein, with at least one sacrificial pre-filter 354; an incubator 356 or heat source for heating an asymmetric membrane that has filtered a sample and is loaded with amplification reagents; and an imager 358 for viewing the amplification products, e.g., amplicon dots, resulting from the amplification reaction in the micropores of the asymmetric membrane.

The hardware included in the system 350 may include standard laboratory devices, in some embodiments. The amplification reagents may include, consist of, or consist essentially of any suitable amplification reagents for initiating and completing an nucleic acid amplification reaction, for example, those described herein. The amplicon imager 358 may include any suitable means for visually inspecting the processed membrane; for example, the imager 358 may include an illumination source, means for dying or marking amplification products in the mixture, and a camera or microscope, such as a fluorescent microscope for capturing images of the illuminated micropores of the membrane presenting any target agent present in a processed sample. For example, the illumination source may be an inexpensive blue (460-470 nm) LED pen used to illuminate the loaded membrane.

The embodiments of the invention(s) may also be illustrated by the following examples, which are provided by way of illustration and are not intended to be limiting.

### EXAMPLES

Asymmetric membrane assay systems were fabricated and used in accordance with methods and systems disclosed herein to detect and quantify Escherichia coli and Salmonella directly in unprocessed environmental samples. In unprocessed environmental sea and pond water with high levels of inhibitors, direct quantification of E. coli and Salmonella was realized with a sensitivity down to single cell and dynamic range of 0.3-10,000 cells/mL.

Two different environments were sampled. Seawater samples were collected from of Santa Monica beach California. Cultured E. coli samples were spiked in with a final concentration of 0.3−1×10⁴ cells/mL and allowed to equilibrate for one hour before analysis. Turtle pond water was collected from the turtle pond at the California Institute of Technology (Caltech) and cultured Salmonella was spiked in with a final concentration of 3−1×10⁴ cell/mL.

For creating cell cultures to test the efficacy of the membrane systems, bacterial strains were obtained from the American Type Culture Collection (ATCC, Manassas, Va.). E. coli (ATCC 10798) was cultivated in Luria-Bertani broth in the shaking incubator for ˜14 hours at 37° C. Salmonella Typhi (CVD 909) was cultivated in tryptic soy broth with 1 mg/L 2,3-dihydroxybenzoate in the incubator for ˜14 hours at 35° C. The concentration of used bacteria suspensions was measured by fluorescence enumeration or standard bacteria culture. For fluorescence enumeration, a bacterial sample was first stained with 1× SYBR Green for 30 minutes, followed by filtration through a commercial PC membrane with 0.2 μm pore size. The cell number was then counted under a fluorescence microscope (Leica DMi8). For bacteria culture assays, bacteria concentrations were quantified by spreading 20 μL of samples on corresponding agar plates, incubating for 12 hours at the respective temperature, and counting the colony-forming units (CFUs). DNA extraction was performed using a commercial beads-beating tube (GeneRite, NJ, USA) or using PureLink DNA extraction kit (Thermo Fisher Scientific) following their instructions.

Asymmetric membranes as described in connection with FIGS. 8a-e were prepared in accordance with the method of FIG. 5. Each asymmetric membrane with a sacrificial PC membrane (having a 2 μm wide pore size) on top was put into a commercial filter holder, e.g., available from Swinnex of Kent, Wash., and 1-10 mL of environmental sample with spiked bacteria was filtered through it using a syringe pushed manually.

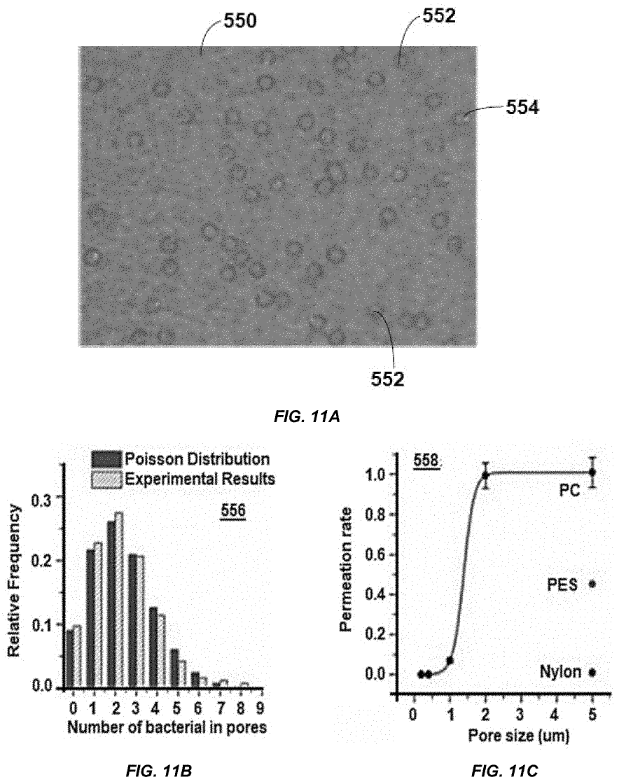

The E. coli permeation rate through the sacrificial pre-filter was tested. As shown in graph 558 of FIG. 11c, the track-etched PC membranes exhibit nearly 100% permeation rate for E. coli (dots on line), even when their pore size was only 2 μm, which was only slightly larger than the size of E. coli (˜1 μm). Upon further decrease of the pore size to 1 μm, the permeation of E. coli was significantly decreased to 5%, exhibiting a perfect cut-off curve for bacterial sieving. This sharp cut-off property was indeed a characteristic behavior of isoporous membranes (membranes with highly ordered channels), such as the membranes disclosed herein, since track-etched PC membranes may have ideal cylinder channels arrays and well-defined pore sizes. In contrast, conventional nylon membranes and PES membranes, which have irregular and intercrossed pore structures, show a poor cut-off performance. Bacteria were easily trapped within the pore networks of the nylon and PES membranes even when 5 μm pore size membranes were used (see FIG. 11c). The sharp cut-off provided by PC membranes also offer the opportunity to collect bacteria/viruses/exosomes at different layers, when two or more membranes with different channel sizes are connected in sequence.

Examples of the asymmetric membrane 400 were used for the filtration of an E. coli sample using a syringe pushed by hand. Due to the high density of microchannels and nanochannels, water passed through the membrane rapidly, and a 1 mL sample was filtered within five seconds. The air in the syringe behind the sample solution pushed all the sample out of asymmetric membrane without a dead volume. Meanwhile, the numerous parallel nanochannels in the asymmetric membrane also alleviated clogging, as the occlusion of any single nanopore resulted in the diversion of the flow to nearby pores.

After filtration, E. coli were randomly captured inside each micropore, while proteins, organics, nucleic acid, ions and other small molecules passed through the nanochannels and were washed away. FIG. 11a shows stained E. coli (bright dots) 554 within the circular micropores 552 of the asymmetric membrane 550 following filtration. All the bacteria were captured and distributed randomly inside the micropores. No bacteria were found outside the pores, even if a relatively high concentration of E. coli was used, e.g., 20000 cell/mL. At this concentration, an average of 2.2 E. coli were trapped in each individual micropore, and the statistic number of E. coli in each pore also fit well with Poisson distribution (see graph 556 FIG. 11b).

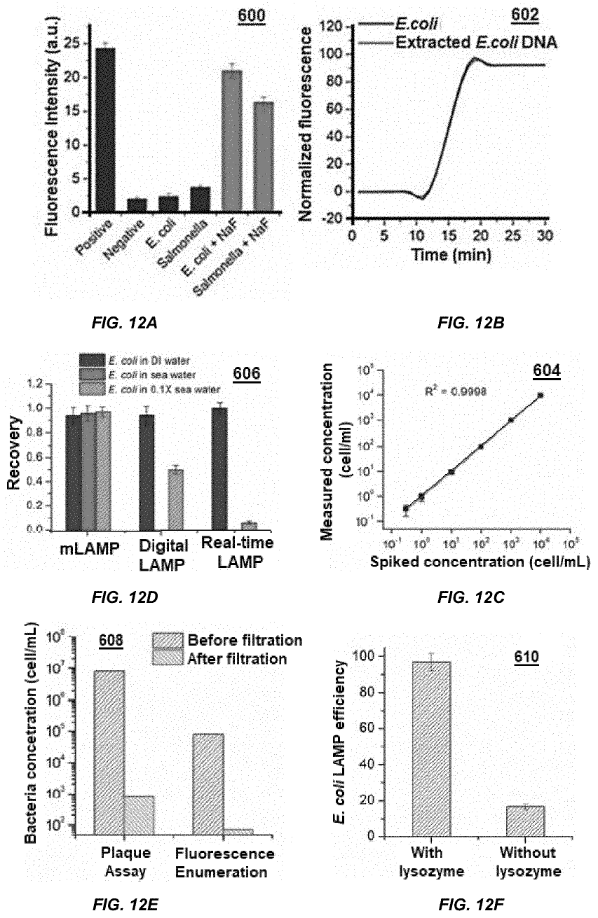

To test the capture efficiency of the membranes, the concentration of E. coli was measured in the original sample, as well as in the filtrate passed through the membrane, by standard bacteria culture and fluorescence enumeration. Results showed that nearly 99.9% of E. coli were captured on the membrane (graph 608 of FIG. 12e). Graph 608 shows a comparison of bacteria capture between a conventional plaque assay and the fluorescence enumeration offered by the asymmetric membrane assay system. This excellent capture efficiency resulted from the outstanding size exclusion and electrostatic repulsion of the nanochannels, even under high flow rates.

After filtration, the sacrificial pre-filter membrane was removed, and 25 μL of modified LAMP mix was added on the top of asymmetric membrane to load inside each micropore of asymmetric membrane for in situ E. coli LAMP. Each micropore was filled with about 13 μL of sample solution/LAMP mix. Due to the capillary forces, the micropores were easily wetted.

The modified LAMP or modified reverse transcription-LAMP reagents included NaF and lysozyme. LAMP was used because it is fast and robust, without the need for thermal cycling. However, as opposed to PCR, which applies a pre-heating (95° C.) step to denature proteins or lyse cells, the Bst polymerase used in the LAMP cannot withstand high temperature. Therefore, single E. coli LAMP in ultrasmall nanoreactor could be easily inhibited in some circumstances. To overcome this, a modified LAMP assay was used for one-step single bacteria LAMP within each pore.

In some embodiments using LAMP, the 25 μL of an example modified LAMP mix for digital single bacteria LAMP contained 1×isothermal buffer, 6 mM total MgSO₄, 1.4 mM dNTP, 640 U/mL Bst 2.0 WarmStart polymerase, 1.6 uM FIB and BIP, 0.2 uM F3 and B3, 0.8 uM LF and LB, 1.5 mg/mL BSA, 50 μM calcein, 1 mM MnCl₂, 2 mM NaF and 0.1 mg/mL lysozyme.

For embodiments using RT-LAMP, WarmStart RTx Reverse Transcriptase was also added to a final concentration of 300 U/mL. The primers for E. coli were designed to be specific to a conserved region on the malB gene, its sequence is as follows:

F3:
(SEQ ID NO: 1)
5-GCCATCTCCTGATGACGC-3;
B3:
(SEQ ID NO: 2)
5-ATTTACCGCAGCCAGACG-3;
BIP:
(SEQ ID NO: 3)
5-CTGGGGCGAGGTCGTGGTATTCCGACAAACACCACGAATT-3;
FIP:
(SEQ ID NO: 4)
5-CATTTTGCAGCTGTACGCTCGCAGCCCATCATGAATGTTGCT-3;
LF:
(SEQ ID NO: 5)
5-CTTTGTAACAACCTGTCATCGACA-3;
LB:
(SEQ ID NO: 6)
5-ATCAATCTCGATATCCATGAAGGTG-3

The primers for Salmonella Typhi were designed to be specific to a conserved region on the STY1607, and its sequence is as follows2:

F3:
(SEQ ID NO: 7)
5-GACTTGCCTTTAAAAGATACCA-3;
B3:
(SEQ ID NO: 8)
5-AGAGTGCGTTTGAACACTT-3;
BIP:
(SEQ ID NO: 9)
5-CCTGGGGCCAAATGGCATTATGCACTAAGTAAGGCTGG-3;
FIP:
(SEQ ID NO: 10)
5-AACTTGCTGCTGAAGAGTTGGACCGAATGACTCGACCATC-3
LF:
(SEQ ID NO: 11)
5-TCGGATGGCTTCGTTCCT-3;
LB:
(SEQ ID NO: 12)
5-CAAGGGTTTCAAGACTAAGTGGTTC-3.

For real-time LAMP performed in a tube for comparison purposes (as opposed to the membrane), the LAMP assay was pre-mixed with 2.5 μL seawater first and incubated at 65° C. using Eppendorf RealPlex2. Fluorescence intensity of the reaction was monitored every minute for 60 minutes. For conventional digital LAMP, the LAMP assay mixture (pre-mixed with 2.5 μL seawater sample) was loaded into each pore of the asymmetric membrane and incubated at 65° C. for 40 minutes for digital LAMP analyses.

To investigate in detail how to overcome LAMP inhibition, real-time LAMP experiments were performed in a tube, followed by polyacrylamide gel electrophoresis. In order to mimic the concentration of bacteria inside the pores, samples with high concentrations of 10⁸ cell/mL were used. The tube reaction for E. coli showed a very weak fluorescence, similar to that of the negative control background, as shown in graph 600 of FIG. 12a. However, gel-electrophoresis results indicated the target E. coli DNA was indeed successfully amplified. A similar phenomenon was also observed when attempting to detect Salmonella (FIG. 12a). Thus, false-negative results were likely caused by inhibitors in the bacterial lysate, which attenuates the fluorescence signal. In the unmodified LAMP assay, calcein-Mn²⁺ indicator was employed for fluorescence reading because of its high signal-to-background ratio. Before amplification, the dye calcein was quenched by the Mn²⁺ and a weak fluorescence was observed. After successful amplification, a large amount of DNA was synthesized, yielding a substantial pyrophosphate as a by-product. The pyrophosphate ions cause the precipitation of Mn²⁺ and the subsequent release of calcein, thus generating a bright fluorescence. The false-negative results may have been attributed to the pyrophosphatase found in bacteria. The pyrophosphatase is a ubiquitous enzyme existing in most organisms for energy metabolism. It is capable of hydrolyzing pyrophosphate ions to phosphate ions, and thus Mn²⁺ will no longer be precipitated. Therefore, the fluorescence of calcein was always quenched. This assumption was confirmed by the observation that no turbidity was observed for bacteria LAMP, although its DNA was successfully amplified. The activity of pyrophosphatase can be inhibited by fluoride ions. As shown in FIG. 12, fluorescence was restored for E. coli and Salmonella samples after including 2 mM NaF into the LAMP reaction to create a modified LAMP, which fluorescence is nearly 10-fold higher compared to the non-template negative control.

Robust single bacteria LAMP performs better with efficient cell lysis. Lysozyme is known for its ability to degrade the peptidoglycans of bacteria cell wall. However, the presence of lysozyme in the reaction may inhibit the PCR process, and should be removed before amplification. By including lysozyme into the LAMP reaction, the bacterial lysis proceeded simultaneously during the isothermal amplification. Effective lysis was proved by the real-time fluorescence results, which showed a coincident amplification curve and same time-to-detection value for E. coli and its extracted DNA when lysozyme was included (graph 602 of FIG. 12b). Meanwhile, the fluorescence enumeration results also demonstrate that almost all the E. coli disappeared after incubation with lysozyme in the tube at 65° C. However, for a sample containing lower bacterial concentration, lysozyme in conventional assay systems may not work well and lysis efficiency may be decreased. However, the digital asymmetric membrane system disclosed herein overcomes this issue, as each single bacteria is encapsulated inside a small pore, which in effect, creates an ultrahigh concentration within the microchannel, regardless of the bulk bacteria concentration.

In some embodiments, a modified LAMP mix including 2 mM NaF and 0.1 mg/mL lysozyme was loaded onto the asymmetric membrane, for digital E. coli LAMP. Modified LAMP was successfully performed on the membrane. The micropores with target bacteria inside generated a bright fluorescence, while those without target bacteria showed a weak background signal. The concentration of target bacteria in the sample can be obtained by direct counting of the positive pores and calibrated by Poisson distribution. The success rate for single E. coli LAMP was as high as 97% (graph 610 of FIG. 12f). The E. coli LAMP efficiency was calculated by measuring the number of stained E. coli on the membrane and the number of positive pores on the membrane. Poisson distribution was also introduced for calibration.

Following loading of the modified LAMP reagents, the wetted asymmetrical membrane was then sealed between two pieces of PDMS film. The membranes were incubated at 65° C. on a hotplate (MJ Research PTC-100, Watertown, Mass.) for 40 minutes. During 65° C. incubation, each pore of the asymmetric membrane functioned as an individual nanoreactor for template amplification, generating a bright fluorescence if a target bacterium was inside a microchannel. Subsequently, the top PDMS was peeled off, followed by adding mineral oil and a frame-seal (Bio-Rad, Hercules, Calif.) to cover the whole membrane. After amplification, the fluorescence images of the membrane were taken by fluorescence microscope (Leica DMi8) using 4×objective. Positive pores were counted using ImageJ (NIH) software and calibrated by Poisson distribution. The total number of pores can be also counted using ImageJ since the negative one also shows a weak fluorescence. However, in this experiment, the total number of pores was estimated based on porosity (1×10⁴ pores/cm²). Each sample was tested at least three times.

By direct counting of positive micropores, absolute quantification of E. coli and Salmonella in unprocessed seawater and pond water samples was achieved within one hour, with a dynamic range from 0.3 to 10,000 cell/mL, even though high levels of inhibitors were present in the samples. In contrast, direct bacteria detection in these environmental samples by conventional methods completely failed.

The results above demonstrate that the asymmetrical membrane can be applied for bacterial capture, concentration, purification and homogeneous partition via a fast one-step filtration process. By comparison, with conventional droplet-based assays, cell encapsulation requires several hours, especially for large sample volumes, causing cell sedimentation, protein inactivation or cell damage. The asymmetric membrane filtration examples disclosed herein were completed within five seconds, which significantly reduces the waiting time and circumvents the problems with known assays.

Additionally, in known assays, nucleic acid amplification in the ultra-small chambers, especially with nanoporous structures, is particularly challenging due to severe adsorption of macromolecules or DNA. However, digital nucleic acid amplification was successfully performed in the microfluidic and nanofluidic partitioned asymmetric membrane system with a high density of nanochannels, as disclosed herein. The disclosed asymmetric membrane provides a digital nucleic acid amplification in a nanofluidic partitioned system with a high density of nanochannels. The underlying nanochannels in the partition system offers the opportunities for solution exchange, while keeping single cells or DNA isolated. Since the bacteria were captured inside the pores first and LAMP reagents were loaded subsequently, the lysis process is restricted to each isolated pore, avoiding pre-release of cell information. These results demonstrate the successful one-step single target agent LAMP within each pore using modified LAMP mixture.

Raw environmental samples typically contain a variety of complex chemical and biological components that will affect the LAMP process. Direct detection of trace amounts of bacteria in these unprocessed samples is difficult and challenging. An example of the asymmetric membrane LAMP system (mLAMP) successfully detected and quantified an extremely low concentration of spiked E. coli in a 10 mL environmental sample directly. When analyzing the sample by mLAMP, the large particles, sand, and planktons in the sample were retained by the pre-filter on top of the asymmetric membrane, while the small inhibitory molecules were washed away through the underlying nanochannels. Meanwhile, the trace amount of E. coli were concentrated in the micropores. Successful quantification of the spiked E. coli in seawater was achieved by mLAMP with a high recovery rate of 95%, as shown in FIG. 12d (graph 606, mLAMP column). The high recovery rate is attributed to full integration of the entire procedure on an asymmetrical membrane system, which significantly reduces potential sample loss. Additionally, no inhibition from a complex seawater matrix was observed, as there were no significant differences for E. coli quantification in seawater or in distilled water (p>0.05), as shown by graph 606 of FIG. 12d.

For comparison, digital LAMP was also performed for E. coli quantification in sea water. This shows the need to filter the sample through the asymmetric membrane first, and then add the LAMP reagents into the microchannels. The results of this comparison are also shown in graph 606. In this test, 22.5 μL LAMP reagent was mixed with 2.5 μL seawater sample first, and then the mixture was loaded inside the pores of an asymmetric membrane for digital amplification. As seen in FIG. 12d (Digital LAMP column), the LAMP reaction was completely inhibited and not a single positive pore was observed. This effect may be due to the presence of high levels of inhibitors (heavy metals or organic matters) in seawater. It should be noted that in this case, the concentration of inhibitors was already diluted 10-times by the LAMP reagents. The inhibition effect is still significant when a further diluted seawater sample (10-times dilution, abbreviated as 0.1×) was used. Only 50% of pores show successful single bacteria LAMP and the observed final fluorescence was lower than normal.

A severe inhibition pattern was also observed for real-time LAMP performed in a tube (real-time LAMP column in graph 606). Due to the poor sensitivity of real-time LAMP, a high concentration of E. coli (5×10⁴ cells/mL) was spiked in the sample. However, the LAMP reaction was still totally inhibited when raw seawater was used (FIG. 12d, Real-time LAMP Column). When a 10-times diluted seawater sample was used, the fluorescence appeared, but with a significant time delay. This delayed amplification resulted in an increased time-to-detection value, and therefore, underestimated the target concentration in the sample.

The foregoing results of alternative experimental LAMP methods demonstrate the excellent performance of the disclosed mLAMP in terms of anti-inhibition for digital bacteria detection in complex fluid samples. mLAMP exhibits excellent performance towards absolute quantification of E. coli at extremely low concentrations, ranging from 0.3 to 10,000 cells/mL, in seawater, with single-cell sensitivity. As shown in FIG. 12c, with more E. coli in the sample, the membrane shows more positive pores. A good linear correlation was observed between the detected absolute number of E. coli and the actual number of cells spiked into the sample (FIG. 12c). Since there is a large error for preparing a single cell in the sample, the lower detection limit (LDL) is defined as the concentration which would have a 95% chance of having at least one bacterium in the sample and equals the concentration of three bacteria per sample. The LDL in this case was 0.3 cell/mL. At this concentration, there were around three positive pores visible on the whole membrane, corresponding to three bacteria in the 10 mL sample.

In addition, the detection of pathogenic Salmonella in turtle pond water was also demonstrated by membrane-based RT-LAMP (mRT-LAMP). Reptiles, like turtles, may carry Salmonella bacteria, which cause diarrhea, stomach pain, nausea, vomiting, fever and headaches. Indeed, the multistate outbreak of Salmonella in the United State during 2015 and 2017 was linked to the contact with turtles carrying Salmonella. Samples were collected from the California Institute of Technology (Caltech) turtle pond. The turtle pond water was more turbid with suspended green algae and mud. These particles were successfully removed by the pre-filter and nanochannels. Primers specific to the gene marker STY1607 were used to detect the corresponding mRNA, as well as DNA. Due to the variations of mRNA copies from cell to cell, it is hard to quantify target cells by detecting the number of mRNAs. However, mRT-LAMP circumvents these difficulties, since each Salmonella bacterium was encapsulated inside a single pore, and thus, the contained nucleic acids, no matter how many, were amplified, resulting in a bright fluorescence. Absolute quantification of spiked Salmonella in pond water was realized for 3-10,000 cells/mL.

The example asymmetrical membrane was capable of bacteria capture, concentration, purification, partition, lysis and digital LAMP without off-membrane sample treatments. Even in unprocessed environmental sea and pond water with a high level of inhibitors, direct quantification of E. coli and Salmonella was realized with a sensitivity down to single cell and dynamic range of 0.3-10,000 cells/mL. Furthermore, the novel membranes are inexpensive (less than 0.1 US dollar) and easily prepared on a large scale. Therefore, they can be thrown away (disposable) after each use, avoiding subsequent LAMP contamination.

Compared with other digital single cell detection methods, mLAMP exhibits many advantages: (i) ten milliliter of samples can be processed on the membrane within seconds, while substantially reducing consumption of precise bioreagents; (ii) all assay steps including bacteria capture, concentration, purification, partition and digital LAMP are integrated onto a single piece of membrane without the need for off-membrane sample treatments. This significantly reduces potential sample loss and simplifies the entire procedure; (iii) with a modified LAMP assay, mLAMP could quantify bacteria at concentrations down to 0.3 cells/mL in unprocessed environmental samples within one hour, even though a relatively high level of inhibitors were present; (iv) tests may be performed using low-cost and disposable commercial membranes without requirement for elaborate chip fabrication or material design; and (v) no complicated or expensive laboratory hardware is required for field analyses.

The disclosed asymmetric membrane system offers fast and low-cost digital quantification, single cell analysis, and other biochemical assays with high throughput. In some embodiments, the membrane may be directly sealed by an adhesive film and imaged by a smartphone to increase the system simplicity for point-of-care diagnostics. In some embodiments, the asymmetrical membranes may also be integrated into a digital membrane system, for example, a nanopore-based DNA sequencing, DNA translocation, molecular exchange, cell electroporation or cell lysis system. In some embodiments, the asymmetric membrane may be paired with paper-based analytical devices for complex sample manipulation and detection. The heterogeneous membrane may serve as an ideal low-cost and simple platform for the rapid detection and analysis of any markers in biological samples, including nucleic acids, bacteria, circulating tumor cells, stem cells, exosomes, viruses, and proteins.

Although disclosed embodiments show the asymmetric membrane as being a planar, round disk shape, the asymmetric membrane may have any suitable shape or curvature, for example the membrane may square, rectangular, triangular or the like, and it may be flat or alternatively curved to any appropriate 3D shape.

Although certain exemplary embodiments disclosed herein may focus on the target agent being microbial pathogens (e.g., E. coli, Salmonella) in environmental water, the systems, membranes, and methods disclosed herein can also be adapted for the detection, quantification, and/or monitoring of other microorganisms, cells, or target nucleic acid in water or food samples in other settings. For example, the systems, membranes, or methods herein may be adapted to microorganism/cell/DNA/RNA detection and quantification in any suitable sample, for example, a gas or combination or gases, fluid, solid, combination of the foregoing, a water or food sample, or a biological sample such as a bodily fluid or matter (e.g., saliva, feces, urine, and blood) with simple sample pretreatment (e.g., target DNA/RNA extraction and/or purification).

The foregoing description is illustrative and not restrictive. Although certain exemplary embodiments have been described, other embodiments, combinations and modifications involving the invention will occur readily to those of ordinary skill in the art in view of the foregoing teachings. Therefore, this invention is to be limited only by the following claims, which cover at least some of the disclosed embodiments, as well as all other such embodiments and modifications when viewed in conjunction with the above specification and accompanying drawings.

What is claimed is:

1. A method of detecting a target agent in a sample suspected of containing the target agent, comprising:
filtering the sample to remove particles larger than the agent to produce a pre-filtered sample;
passing the pre-filtered sample through a plurality of first channels formed in a membrane, the first channels forming a corresponding plurality of pores on an exposed surface of the membrane for admitting the sample into the first channels, wherein each of the first channels has a predetermined width configured to admit a predetermined number of the target agent into each of the first channels;
passing the sample output from the first channels through a plurality of second channels formed in the membrane and connected to the first channels, wherein the second channels are configured to trap one or more individuals of the target agent in the first channels and allow one or more other constituents of the pre-filtered sample to pass through the second channels out of the membrane;
applying one or more reagents into the first channels through the pores to cause an amplification reaction involving the agent; and
detecting the presence or absence of one or more amplification products trapped within the first channels that are produced as a result of the amplification reaction amplifying a nucleic acid of the target agent if the target agent is present in any of the first channels, wherein the presence of the amplification products is indicative of the presence of the target agent in the sample and the absence of the amplification products is indicative of the absence of the target agent in the sample.

2. The method of claim 1, further comprising:
passing substantially all of the sample through the first channels and second channels of the membrane prior to applying the reagents into the first channels.

3. The method of claim 1, wherein the reagents include reagents selected from the group consisting of isothermal amplification (LAMP) reagents and reverse transcription-LAMP.

4. The method of claim 3, wherein the reagents further include NaF and lysozyme.

5. The method of claim 1, wherein the amplification reaction is selected from the group consisting of isothermal amplification (LAMP), modified LAMP, reverse transcription-LAMP (RT-LAMP), modified RT-LAMP, polymerase chain reaction (PCR), reverse transcription PCR (RT-PCR), quantitative PCR (qPCR), and reverse transcription qPCR (RT-qPCR).

6. The method of claim 1, further comprising:
after passing the sample through the first channels and the second channels of the membrane and applying the reagents, sealing the exposed surface of the membrane and heating the membrane.

7. The method of claim 1, further comprising:
imaging the exposed surface of the membrane with a camera or a fluorescence microscope to visually detect the presence or absence of amplification products in the first channels.

8. The method of claim 1, wherein the sample is selected from the group consisting of environmental water, processed water, bodily fluid, urine, blood, feces, and any combination of the foregoing.

9. The method of claim 1, wherein the target agent is a virus, protozoa, fungi, cell, or bacteria.

10. A method of manufacturing a composite membrane for detecting a target agent, comprising:
providing a first track-etched membrane having a plurality of first channels passing therethrough between a first surface of the first membrane and a second surface of the first membrane, wherein each of the first channels has a predetermined width configured to admit a predetermined number of the target agent into each of the first channels;
placing a second track-etched membrane on either the first surface or second surface of the first track-etched membrane, the second track-etched membrane having a plurality of second channels passing therethrough, wherein each of the second channels has a width smaller than the predetermined width of the first channels; and
with the second track-etched membrane emplaced on the first track-etched membrane, heating the first and second track-etched membranes to bond them together.

11. The method of claim 10, wherein the first track-etched membrane and the second track-etched membrane are made of the same polymer.

12. The method of claim 11, wherein step of heating includes heating the first track-etched membrane and the emplaced second track-etched membrane to a temperature above the glass-transition temperature of the polymer.

13. The method of claim 11, wherein the polymer is a polycarbonate.

14. The method of claim 11, wherein the first channels are microchannels having a uniform width greater than 1 μm and the second channels are nanochannels having a uniform width less than 400 nm.

15. A membrane for detecting a target agent in a sample suspected of containing the target agent, the membrane comprising:
a first layer having a plurality of first channels passing therethrough, the first channels forming a corresponding plurality of pores on an exposed surface of the first layer for admitting the sample into the first channels, wherein each of the first channels has a predetermined width configured to admit a predetermined number of the target agent into each of the first channels; and
a second layer contacting first layer, the second layer having a plurality of second channels passing therethrough and connecting with the first channels of the first layer, wherein the second channels are configured to retain one or more individuals of the target agent in the first channels and pass one or more other constituents of the sample out of the membrane though an exposed surface of the second layer.

16. The membrane of claim 15, wherein the first channels are microchannels having a uniform width greater than 1 μm and the second channels are nanochannels having a uniform width less than 400 nm.

17. The membrane of claim 15, wherein the first layer and the second layer are each about 25 μm thick.

18. The membrane of claim 15, included in a system for detecting the target agent in the sample suspected of containing the target agent, the system further including:
a sacrificial filter configured to pre-filter the sample to remove particles larger than the agent prior to the sample being passed through the membrane.

19. The membrane of claim 15, included in a system for detecting the target agent in the sample suspected of containing the target agent, the system further including:
one or more amplification reagents; and
means for applying the amplification reagents into the first channels of the membrane.

20. The membrane of claim 19, wherein the amplification reagents include:
reagents selected from the group consisting of isothermal amplification (LAMP) reagents and reverse transcription-LAMP reagents; NaF; and Lysozyme.
